(19) United States
(12) Patent Application Publication
(10) Pub. No.: US 2010/0144024 A1
(43) Pub. Date: Jun. 10, 2010

(54) REPLICA MOULDING OF MICROSTRUCTURES FOR SUPPORTING MICROSCOPIC BIOLOGICAL MATERIAL

(75) Inventors: Daniel Day, Hawthorn (AU); Min Gu, Doncaster (AU)

Correspondence Address: SEED INTELLECTUAL PROPERTY LAW GROUP PLLC, 701 FIFTH AVE, SUITE 5400, SEATTLE, WA, 98104, US

(73) Assignee: SWINBURNE UNIVERSITY OF TECHNOLOGY, Hawthorn (AU)

(21) Appl. No.: 12/517,058

(22) PCT Filed: Nov. 30, 2007
(86) PCT No.: PCT/AU2007/001853

§ 371 Date: Jan. 25, 2010

(30) Foreign Application Priority Data
Dec. 1, 2006 (AU) 2006906741

(51) Int. Cl. C12M 1/22; B29C 39/02; B01L 3/00

(52) U.S. Cl. 435/305.1; 264/225; 422/99

(57) ABSTRACT

A method of replica moulding microstructures, the method including forming a negative master of at least one microstructure configured to support microscopic biological material, casting a flowable polymeric material onto the negative master, placing a substrate against the flowable polymeric material and the negative master, allowing the flowable polymeric material to solidify in the negative master and on the substrate, and separating the substrate and the solidified polymeric material from the negative master, thereby leaving a positive replica of the at least one microstructure on the substrate.

### FIELD OF THE INVENTION

The present invention relates to replica moulding of microstructures for supporting microscopic biological material.

### BACKGROUND OF THE INVENTION

Microscopic biological material, such as cellular material, is conventionally retained for observation on glass cover slips or in cell culturing plates. Cover slips are low cost but their flat surfaces are ill suited to retaining microscopic biological material. Cell culturing plates retain microscopic biological material in arrays of macroscopic wells which are larger than the typical field of view for live cell imaging.

A need therefore exists for a low cost, generic technology for supporting microscopic biological material.

### SUMMARY OF THE INVENTION

According to the present invention, there is provided a method of replica moulding microstructures, the method including forming a negative master of at least one microstructure configured to support microscopic biological material, casting a flowable polymeric material onto the negative master, placing a substrate against the flowable polymeric material and the negative master, allowing the flowable polymeric material to solidify in the negative master and on the substrate, and separating the substrate and the solidified polymeric material from the negative master, thereby leaving a positive replica of the at least one microstructure on the substrate.

The at least one microstructure can be selected from a microgrid, a microwell, a microplatform, and combinations thereof.

The flowable polymeric material can be poly(dimethylsiloxane) (PDMS).

The substrate can be a cover slip or a microscope slide.

The present invention also provides a device for supporting microscopic biological material made by the above replica moulding method.

The device can be a cell culturing plate or a microwell plate.

### BRIEF DESCRIPTION OF THE DRAWINGS

The present invention will now be described by way of non-limiting example only with reference to the accompanying drawings in which:

FIG. 1 is a flow chart of a method of replica moulding of microstructures for supporting microscopic biological material; and

FIGS. 2(a) to 2(d) are scanning electron microscope (SEM) images of different microstructures made by the replica moulding method.

### DETAILED DESCRIPTION

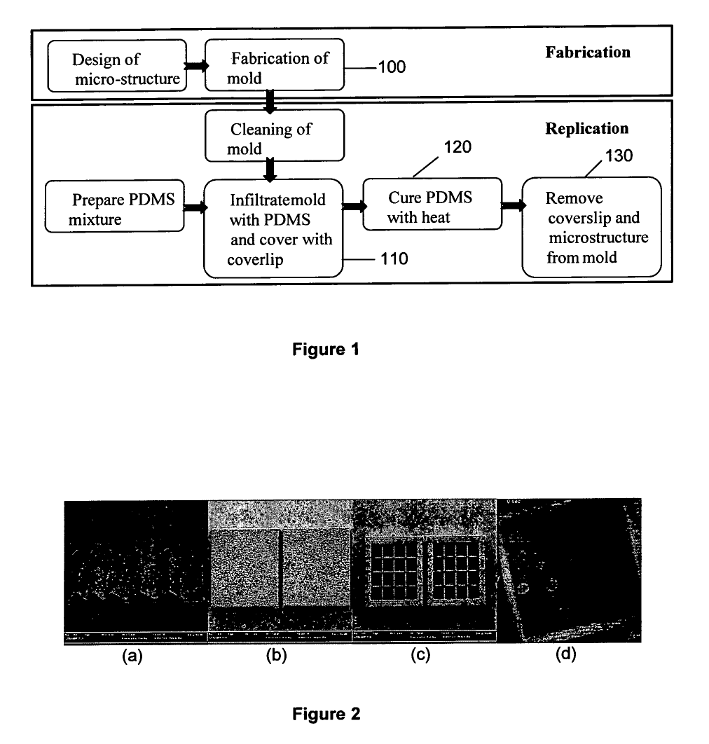

FIG. 1 illustrates a replica moulding method of one embodiment of the invention. The method starts at step 100 by forming a negative master mould of one or more microstructures configured to support microscopic biological material, for example, cells or cellular material. The microstructures can be a microgrid, a microwell, a microplatform and combinations thereof. Other equivalent microstructures designed for supporting microscopic biological material can also be used. The negative master can be made of, for example, poly(methyl methacrylate) (PMMA). Other equivalent materials may also be used for the negative master. The negative master can be fabricated by etching out the inverse of the final microstructure using amplified femtosecond pulse laser (Spitfire, Spectra Physics). Other equivalent fabrication techniques can also be used. After fabrication, the negative master is cleaned.

Next at step 110, liquid poly(dimethylsiloxane) (PDMS) is cast onto the negative master. Other equivalent casting materials may also be used. After the PDMS has infiltrated the negative master, the PDMS in the negative master is covered with a substrate, for example, a cover slip, a glass microscope slide, a silicon wafer, etc.

At step 120, the negative master is heated on a hotplate at 85° C. for 20 minutes to allow the PDMS to cure and solidify on the negative master and the substrate. The substrate and the solidified PDMS are separated from the negative master at step 130, thereby leaving a positive PDMS replica of the microstructure on the substrate.

FIGS. 2(a) to 2(d) illustrate different microstructures made by the above replica moulding method 100 for use in biological research. For example, the positive replica PDMS microplatforms of FIGS. 2(a) and 2(b) can be used to investigate cellular mechanics, while the positive replica PDMS microgrid and microwell of FIGS. 2(c) and 2(d) can be used to trap and observe cellular activity within a confined environment. Embodiments of the invention can be implemented as devices for supporting microscopic biological material, for example, cell culturing plates or microwell plates.

Embodiments of the invention therefore provide a low cost, generic technology for supporting microscopic biological material.

The embodiments have been described by way of example only and modifications are possible within the scope of the claims which follow.

1. A method of replica moulding microstructures, the method including forming a negative master of at least one microstructure configured to support microscopic biological material, casting a flowable polymeric material onto the negative master, placing a substrate against the flowable polymeric material and the negative master, allowing the flowable polymeric material to solidify in the negative master and on the substrate, and separating the substrate and the solidified polymeric material from the negative master, thereby leaving a positive replica of the at least one microstructure on the substrate.

2. A method according to claim 1, wherein the at least one microstructure is selected from a microgrid, a microwell, a microplatform, and combinations thereof.

3. A method according to claim 1 or 2, wherein the flowable polymeric material is poly(dimethylsiloxane) (PDMS).

4. A method according to any preceding claim, wherein the substrate is a cover slip or a microscope slide.

5. A device for supporting microscopic biological material made by a method according to any preceding claim.

6. A device according to claim 5, wherein the device is a cell culturing plate or a microwell plate.
